(19) United States
(12) Patent Application Publication
(10) Pub. No.: US 2013/0344047 A1
(43) Pub. Date: Dec. 26, 2013

(54) Methods And Composition For Treating Heart Failure And Ischemia

(71) Applicant: ABBOTT CARDIOVASCULAR SYSTEMS, INC., Santa Clara, CA (US)

(72) Inventors: Stephen D. Pacetti, San Jose, CA (US); Jeffrey B. Huff, Park Ridge, IL (US); Paul Consigny, San Jose, CA (US)

(73) Assignee: ABBOTT CARDIOVASCULAR SYSTEMS, INC., Santa Clara, CA (US)

(21) Appl. No.: 14/014,659

(22) Filed: Aug. 30, 2013

Related U.S. Application Data
(63) Continuation-in-part of application No. 13/332,509, filed on Dec. 21, 2011, now Pat. No. 8,546,412.

(51) Int. Cl. A61K 35/14; A61K 38/19; A61K 45/06

(52) U.S. Cl. CPC A61K 35/19; A61K 45/06; A61K 38/196; A61K 38/195
USPC 424/93.72

(57) ABSTRACT

Methods and compositions for treating heart failure by administration of beneficial agents to the heart.

### RELATED APPLICATION

This application is a continuation-in-part patent application of co-pending U.S. Ser. No. 13/332,509 filed on Dec. 21, 2011, the content of which is hereby incorporated by reference.

### TECHNICAL FIELD

The subject matter relates to methods and compositions for treating and/or inhibiting heart failure and ischemia.

### BACKGROUND

Heart failure, often referred to as congestive heart failure (“CHF”) is a debilitating, often fatal, disease that is generally defined as the inability of the heart to supply sufficient blood flow to the tissues and organs of the body. In some instances, the cardiac output is low, and the body becomes congested with fluid. However, fluid overload is not co-terminus with heart failure and some patients may be euvolaemic or dehydrated.

During heart failure, the ventricles of the heart fill with blood, but the heart muscle loses progressively more and more of its ability to cross link actin and myosin fibers over the stretched heart muscle. This causes heart muscle contraction to become less efficient. This reduced contractility also reduces stroke volume. In cardiovascular physiology, stroke volume is the volume of blood pumped from one ventricle of the heart with each beat. Other changes in the heart include: loss of one's cardiac reserve, especially during exercise; increased heart rate, which may be stimulated by increased sympathetic activity to maintain cardiac output; arrhythmias; hypertropy (an increase in physical size) of the myocardium; and enlargement of the ventricles, which contributes to enlargement and increasingly spherical shape of the failing heart. The increase in ventricular volume can also cause a reduction in stroke volume due to mechanical and contractile inefficiency.

In chronic heart failure, the reduced cardiac output can cause various changes in the rest of the body, such as decreased arterial blood pressure, increased sympathetic stimulation, which may cause increased secretion of vasopressin, fluid retention and consequently increased blood pressure. Other changes include reduced perfusion to organs such as the kidneys, which may cause secretion of renin, an enzyme that catalyses the production of the vasopressor angiotensin. Angiotensin and its metabolites can cause further vasocontriction, and stimulate increased secretion of aldosterone, which can further promote salt and fluid retention at the kidneys, also increasing the blood volume and blood pressure.

Chronic high levels of hormones such as renin, angiotensin, and aldosterone can negatively affect the myocardium, causing structural remodeling of the heart over the long term. Thus, the maladies of chronic heart failure can be persistent and pervasive.

The National Health and Nutrition Examination Survey (NHANES I) identified the following causes of heart failure: ischemic heart disease, hypertension, valvular heart disease, dilated cardiomyopathy, obesity, and diabetes. Ischemic heart disease occurs when vessels carrying blood in the coronary and/or peripheral vasculature are partially or completely blocked. When these vessels are partially blocked, lack of blood flow causes a lack of oxygen supply to the muscle tissue. A lack of oxygen supply can cause interference with proper muscle contraction and function. If the vessel is completely blocked, the muscle tissue dies.

In some individuals, blockage of the blood vessel can be partially compensated by natural processes. For example, new vessels can be formed (termed “angiogenesis”) and small vessels can enlarge (termed “arteriogenesis”) to replace the function of the impaired vessels. These new conduits may facilitate restoration of blood flow to the deprived tissue, thereby constituting “natural bypasses” around the occluded vessels.

### SUMMARY

In accordance with the purpose of the disclosed subject matter, a method of reducing heart failure after myocardial infarction is disclosed. In this regard, the method includes obtaining platelets from a donor and increasing the stromal cell derived factor (SDF-1) content of the donor platelets. The donor platelets with increased SDF-1 are administered, directly or indirectly, to the heart of the subject. For example, the donor platelets can be administered to the myocardium or coronary artery by intravenous injection or local delivery though a lumen.

The administered donor platelets can bind to the internal surfaces of an injured artery within the infarct zone. Alternatively, the administered donor platelets may aggregate to form microvascular obstructions in the infarct zone. In either of the above cases, the net result is increased platelet deposition and thereby increased SDF-1 delivery by these loaded platelets to the injury site. Further, the SDF-1 delivered by the donor platelets can stimulate the recruitment and sequestration of progenitor cells and replace myocardial cells and promote revascularization of the infarct site, thereby promoting healing and myocardial wall thickening and inhibiting cardiac wall expansion that leads to heart failure.

The SDF-1 content can be increased by electroporating the donor platelets in the presence of SDF-1. If desired, the platelets can be activated with a cytokine, such as thrombopoietin prior to administration.

In another aspect, a composition includes a platelet and an activator disposed within the platelet, the activator having a property that induces or modulates therapeutic angiogenesis. Examples include, but are not limited to, a composition comprising a platelet in a non-transformed state that induces and/or modulates therapeutic angiogenesis and/or a platelet that is modified such that a treatment agent, such as a specific binding treatment agent (as defined herein), is contained within the platelet such that, through degradation of the platelet, the treatment agent is released at a treatment site and induces or modulates therapeutic angiogenesis.

In another embodiment, a method is disclosed. The method includes introducing a treatment agent comprising a platelet at a treatment site selected for therapeutic angiogenesis. A suitable treatment site includes a blood vessel (e.g., the wall of a blood vessel) or beyond the wall of a blood vessel. The treatment site may be established by positioning a delivery device such as a catheter at or adjacent to the treatment site.

In a further embodiment, a kit is disclosed. One example of such a kit is a kit comprising an injectable composition having the property of inducing therapeutic angiogenesis such as a composition of a plurality of platelets that may or may not be modified.

### BRIEF DESCRIPTION OF THE DRAWINGS

FIG. 1 schematically illustrates a perspective and cross-sectional view of a blood vessel;

FIG. 2 schematically illustrates a planar cross-sectional view of components of a coronary artery network;

FIG. 3 is a flow chart of a technique to form a treatment agent comprising a blood platelet;

FIG. 4 is a simplified cross-sectional view of an embodiment of a substance delivery apparatus in the form of a catheter assembly having a balloon and a therapeutic substance delivery assembly;

FIG. 5 schematically illustrates a planar cross-section of the substance delivery apparatus of FIG. 4 through line A-A′;

FIG. 6 schematically illustrates a planar cross-section of the substance delivery apparatus of FIG. 4 through line B-B′;

FIG. 7 schematically illustrates a cross-sectional view of the distal section of the substance delivery apparatus of FIG. 4 with the balloon in an undeployed configuration;

FIG. 8 schematically illustrates a cross-sectional view of the distal section of the substance delivery apparatus of FIG. 4 with the balloon in a deployed configuration;

FIG. 9 schematically illustrates the left coronary artery network having a catheter assembly introduced therein; and

FIG. 10 presents a block diagram for introducing a treatment agent.

The features of the described embodiments are specifically set forth in the appended claims. However, the embodiments are best understood by referring to the following description and accompanying drawings, in which similar parts are identified by like reference numerals.

### DETAILED DESCRIPTION OF EXEMPLARY EMBODIMENTS

### Overview

The exemplary embodiments relate to the treatment of heart failure. Heart failure, as used herein, is defined as an inability of the heart to supply sufficient blood flow to meet the needs of the body. In some embodiments, the treatment includes locally administering a prodrug to the heart muscle, for example via the pericardium. The prodrug converts to an active drug in situ. The term “prodrug” as used herein means a precursor of a drug that undergoes a chemical conversion before becoming an active pharmacological agent. The term “active drug” as used herein means an active pharmacological agent. In some embodiments, the treatment includes administering platelets having increased content of SDF-1.

### Treatments of Heart Failure

In one aspect, a method for treating heart failure is disclosed. As used herein a “subject” is an individual in need of said treatment. The method includes administering a prodrug to the heart of the subject; and converting or causing conversion of the prodrug to an active drug in situ at the heart. In this regard, the heart-failure outcome can be prevented and blood-pumping effectiveness improved by the local delivery of drug to the heart.

In one embodiment, the prodrug is lipophilic. The lipophilic prodrug is advantageous due to the typically high lipid content of the pericardium. The fatty deposit region of the pericardium presents an ideal environment to deliver and concentrate a lipophilic prodrug. Being more lipophilic, the prodrug will have a longer residence time in the fatty tissue of the pericardium than the parent drug. One measure of lipophilicity is the n-octanol/water partition coefficient. Preferably, the lipophilic prodrug has an octanol/water partition coefficient at least twice as large as that of the parent drug. After local delivery, the lipophilic prodrug is available for enzymatic conversion or other catalyzed conversion to an active form of the drug in the vicinity of the heart muscle.

Several classes of drugs offer potential benefit when administered locally for congestive heart failure. One such class are the statins. Rosuvastatin and a hydrophobic prodrug lauryl alcohol derivative of rosuvastatin have the following structures:

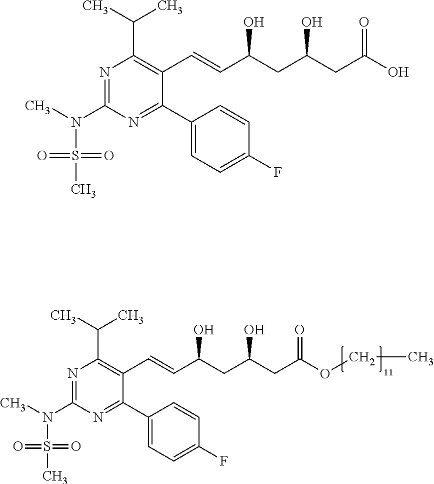

In vivo, the lauryl alcohol ester would slowly hydrolyze, generating the parent drug. In another embodiment, the angiotensin converting enzyme (ACE) inhibitor Captopril is converted to a lauric acid thioester prodrug derivative with greater lipophilicity than Captopril.

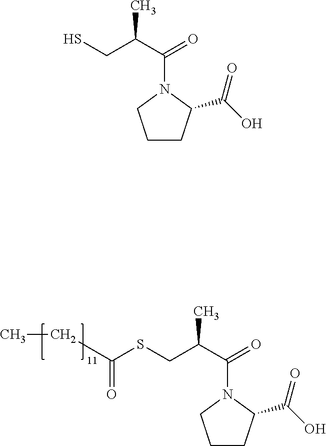

In vivo, the thioester will hydrolyze more rapidly than an aliphatic ester, liberating the parent drug molecule. In one embodiment, lipophilic groups can be attached to an active drug molecule, such as but not limited to alkyl chains, polyethers, branched hydrocarbons, or other suitable chemical moieties. The attachments of lipophilic groups preferably include various reversible attachment moieties such as esters, amides, peptides, disulfides, thioesters, ketals, acetals, orthoesters, N-hydroxyy succinimidyl or other linkers that are subject to nucleophilic attack, hydrolysis, reduction, oxidation, or other enzyme- or naturally-catalyzed reactions to release the active drug moiety from the lipophilic region of the heart and induce distribution within the target tissue for a therapeutic effect.

In one embodiment, the prodrug is administered to the pericardial sac of the heart. The drug may be introduced into the pericardial sac via a subxyphoid injection. An alternate technique is via a needle injection catheter introduced through the venous system into the right atrium, and then a transatrial wall needle injection into the pericardial sac. In another embodiment, the prodrug is administered directly to the myocardium.

In another aspect, the bioavailability of an active drug for treating heart failure is increased by the local delivery of a prodrug that is converted to an active drug in situ and in particular, can be increased at the site of treatment. In this regard, the toxicity of systemic delivery of active agents can be avoided. Thus, toxicity associated with the active drug is reduced as compared to systemic delivery of the active drug. There are several examples of local delivery of a therapeutic agent in order to avoid systemic effects. One such example is the Gliadel wafer used to treat malignant glioma. This releases carmustine locally, avoiding the higher dose needed if it were administrated systemically and the toxicity of systemic administration. A second example would be the drug eluting stent (DES) used to treat obstructive coronary artery disease. For example, the XIENCE® V drug eluting stent releases everolimus locally to prevent neointimal hyperplasia after stenting, which leads to restenosis. Research has been performed in using systemic administration of drugs such as sirolimus or everolimus in combination with metallic stents. Sufficiently high oral doses of these drugs can reduce restenosis. However, common side effects are nausea, abdominal pain, and bacterial infections.

In another aspect, a method of treating or inhibiting heart failure after an ischemic event, such as myocardial infarction, is disclosed. In this regard, platelets are obtained from a donor and the SDF-1 content is increased. In this regard, the injury site exposes platelet binding sites and releases platelet activators. A cascade effect including recruitment of SDF-1 loaded platelets occurs, which release SDF-1 locally at the site. SDF-1 is a progenitor cell chemo-attractant, thus progenitor cells are also recruited to the injury site. Differentiation into myocardial and vascular cells plus cytokine and growth factor release enhance cell recruitment, proliferation, and matrix deposition; all of which promotes and improves healing of the injury including increasing cardiac wall thickening and contractility, and decreasing occurrences of heart failure.

In accordance with this aspect, modified donor platelets are administered to a subject. The SDF-1 content of the platelets is increased by electroporating the donor platelets in the presence of SDF-1. The electroporation increases SDF-1 content and platelet activation increases surface expression. Other cells that may be recruited to the infarct site include neutrophils and macrophages. These cells can also be electroporated to increase SDF-1. SDF-1 protein can be obtained from various sources including platelets, smooth muscle cells, endothelial cells and macrophages.

The method includes administering the modified platelets to a coronary artery, for example, a coronary artery that supplies an infarcted myocardium. Alternatively, the platelets can be administered into the left ventricle (upstream of the coronary circulation) or by intra-arterial or intravenous injection.

In another embodiment, platelet surface expression of SDF-1 is increased by activation by thrombin or cytokine stimulation by a soluble kit ligand, or thrombopoietin. Surface-bound SDF-1 can bind to CXCR4 receptors on circulating progenitor cells and facilitate cell recruitment. The recruitment of progenitor cells can increase cardiac mass and increase cardiac shortening.

In regard to the method, the modified platelets will bind to the surfaces of injured arteries within the infarct zone or will aggregate to form microvascular obstructions in the infarct zone. The platelet activation within the infarct zone will stimulate the expression of SDF-1 on the surface of the platelet or release into the extracellular fluid. The SDF-1 can stimulate the recruitment and sequestration of progenitor cells to replace myocardial cells and/or promote revascularization of the infarct site. Accordingly, the method described can result in improved myocardial function and reduce the likelihood or forestall the progression of heart failure, in particular after an ischemic event such as myocardial infarction. Further, the mobility of stem cells to the heart can reduce the severity of the heart failure.

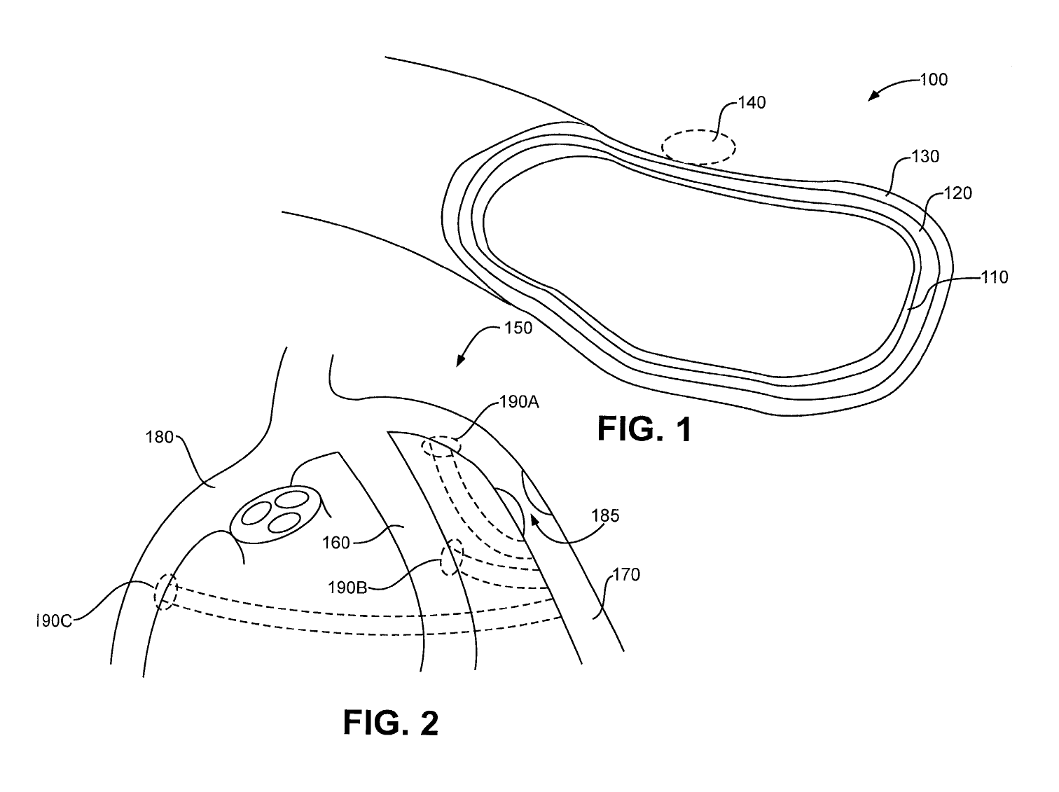

Referring to FIG. 1, a non-diseased artery is illustrated as a representative blood vessel. Artery 100 includes an arterial wall having a number of layers Innermost layer 110 is generally referred to as the intimal layer that includes the endothelium, the subendothelial layer, and the internal elastic lamina. Medial layer 120 is concentrically outward from intimal layer 110 and bounded by external elastic lamina and adventitial layer 130 is the outermost layer. There is no external elastic lamina in a vein. Medial layer 120 (in either an artery or vein) primarily consists of smooth muscle fibers and collagen. Beyond medial layer 120 and adventitial layer 130 lies the extravascular tissue including, adjacent adventitial layer 120 (and possibly including a portion of adventitial layer 130), area 140 referred to as peri-adventitial site (space) or area. Areas radially outward from a peri-adventitial space include connective tissue such as adipose tissue that is most likely located, in terms of areas around the heart, toward the epicardial surface of the heart and myocardial tissue composed of muscle fibers.

FIG. 2 illustrates components of a coronary artery network. In this simplified example, vascular 150 includes left anterior descending artery (LAD) 160, left circumflex artery (LCX) 170 and right coronary artery (RCA) 180. Occlusion 185 is shown in LCX 170. Occlusion 185 limits the amount of oxygenated blood flow through LCX 170 resulting in a condition referred to as ischemia in the tissues distal to the occlusion.

To improve the function of the artery network, it is generally desired to either remove occlusion 185 (for example through an angioplasty procedure), bypass occlusion 185 or induce therapeutic angiogenesis to make-up for the constriction in the ischemic region (e.g., downstream of occlusion 185). FIG. 2 shows therapeutic angiogenesis induced at sites 190A (associated with LCX 170); 190B (associated with LAD 160); and 190C (associated with RCA 180). By inducing therapeutic angiogenesis at sites 190A, 190B, and 190C, permanent revascularization of the network is accomplished, thus compensating for reduced flow through LCX 170. The following paragraphs describe compositions, techniques and an apparatus suitable for inducing therapeutic angiogenesis.

Blood platelets release factors that can induce therapeutic angiogenesis. Blood platelets include cellular elements of blood about two to four microns (gm) in diameter that generally have no nuclei and originate as a fragment of large cells in the bone marrow.

VEGF is a growth factor that stimulates endothelial cell migration, proliferation, and capillary formation. Recent studies have demonstrated that platelets contain VEGF messenger ribonucleic acid (RNA) and VEGF-C and VEGF protein. These proteins are released by platelets activated by agents such as thrombin, collagen, epinephrine, and adenosine diphosphate (ADP). Upon activation, platelets release a number of lipids that are believed to play a role in therapeutic angiogenesis. One such lipid, spingosine-1-phosphate (S1P), is stored in platelet granules and is released upon platelet activation. Other angiogenic platelet lipids include lysophosphatidate (LPA) and phosphatidic acid (PA). These three lipids bind to endothelial differentiation gene (EDG) receptors, a novel family of G-protein-coupled receptors present in endothelial cells. When stimulated, these receptors activate pathways that ultimately result in endothelial cell responses associated with therapeutic angiogenesis (e.g., liberation of endothelial cells from established monolayers, chemotactic migration, chemokinesis, proliferation, and morphogenesis into capillary-like structures).

One advantage to using platelets as a treatment agent for effecting (e.g., inducing and/or modulating) therapeutic angiogenesis is that platelets are present in circulating blood and therefore a patient may be the donor of his/her own platelets. Alternatively, other platelet donors may be accessed via, for example, a blood bank. Platelets typically have a life span on the order of about seven to eight days. Therefore, platelets introduced (i.e., from a donor other than the patient) at a treatment site should not induce an allergic response.

Platelets can effect therapeutic angiogenesis typically by themselves, for example, through cell lysis or by breaking down the cell structure releasing a factor, such as the therapeutic angiogenic treatment agent, therein. Alternatively, the platelets may be modified to include an activator selected for the activator's ability to effect therapeutic angiogenesis.

One way that platelets may be modified to include an activator selected for the activator's ability to effect (induce and/or modulate) therapeutic angiogenesis is by electroporating the blood platelet to allow the introduction of the activator. Recent studies have demonstrated that electroporation can be used to place drugs inside blood platelets. The process generally involves the use of a brief pulse of electricity to increase the permeability of each platelet's membrane and thereby permits a drug to diffuse passively into the platelet cell. Upon cessation of the electric field, the membrane permeability returns to normal and the drug is released only after platelet activation, cell lysis or death. The method has been used to incorporate iloprost into platelets and iloprost-containing platelets have been used to reduce platelet aggregation in balloon-dilated arteries.

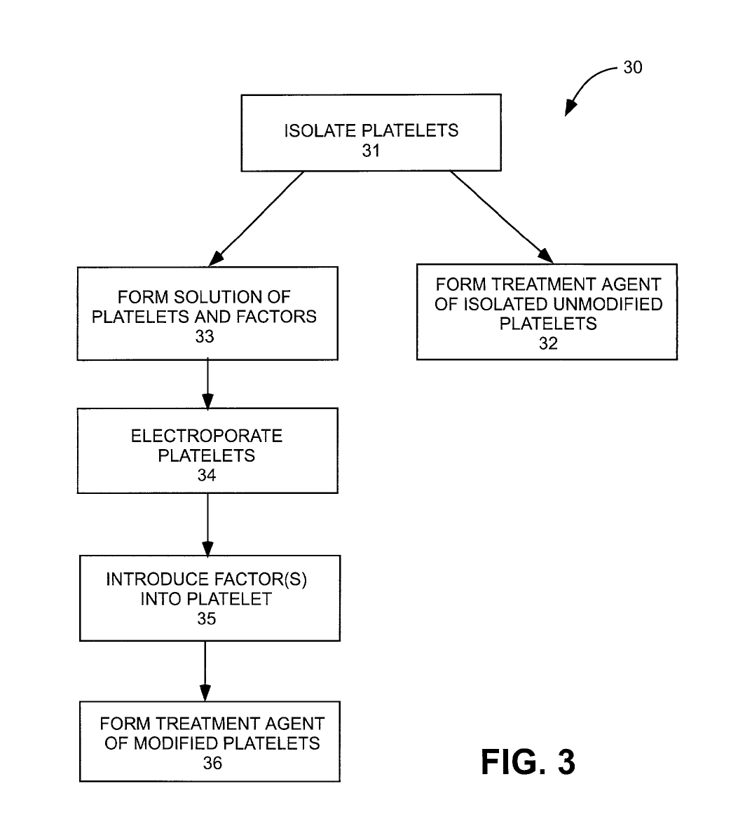

FIG. 3 presents a process flow for forming an amount of blood platelets suitable for use in an amount (possibly in a kit) for effecting (e.g., inducing and/or modulating) therapeutic angiogenesis. Referring to FIG. 3 and process flow 30, in this example, a suitable or desired amount of blood platelets is isolated from a patient's blood or a donor's blood using, for example, centrifugation techniques (block 31). The isolation of blood platelets through centrifugation techniques is known. Such isolated platelets may be used directly as a treatment agent to effect (e.g., induce and/or modulate) therapeutic angiogenesis (block 32). A representative amount or concentration of unmodified blood platelets suitable for effecting therapeutic angiogenesis is on the order of 108 to 1012 platelets per milliliter for a dosage of 0.10 to 1.0 milliliters injected at a treatment site. It is appreciated that in, for example, a technique to effect therapeutic angiogenesis, the amount or concentration may vary considerably. On the one hand, a relatively small amount or concentration may be introduced at a treatment site (e.g., a peri-adventitial space or radially outward from a peri-adventitial space) in an effort to supplement or stimulate a body's own ability to effect therapeutic angiogenesis. On the other hand, blood platelets are a naturally occurring substance in mammalian (e.g., human) bodies and therefore risks of overloading or overdosing are minimal.

Referring to FIG. 3, rather than introducing unmodified platelets to effect therapeutic angiogenesis, the platelets may be modified to include a factor that has a property that effects therapeutic angiogenesis. One way to modify the isolated platelets is through electroporation. The platelets may be electroporated using a mammalian cell electroporator, such as an Eppendorf Multiporator from Brinkman Instruments of Westbury, N.Y. or EASYJECT PLUSTM® from Equibio of Ashford, Middlesex, United Kingdom. Methods have been developed for platelet electroporation. Such methods include forming a solution of isolated platelets and factors (factors that have a property that affects (e.g., induces and/or modulates) therapeutic angiogenesis) so as to create a concentration gradient between the solution of factors and the cytoplasm of the platelets (block 33).

Following the formation of the solution of platelets and factors, the solution is subjected to an electric field in an electroporation process (block 34). In one example, the solution is subjected to a field strength of an electroporator of on the order of six to eight kilovolts (kV) per centimeter with a pulse width of 15 to 18 microseconds. The pulse (e.g., a small brief electrical pulse) is thought to reversibly disrupt the cell membrane. The disruption creates small openings in the membrane to allow molecules to enter the cytoplasm of the platelet cell.

Following electroporation of the platelet cells, factors may be introduced into the platelets (block 35). In one example, the concentration gradient of the solution may be such that a factor contained within the solution enters the small opening(s) in the platelet membrane thereby placing desired factors into the platelet.

In the description of forming platelets activated with an introduced (e.g., foreign) factor selected for a property that affects therapeutic angiogenesis, it is appreciated that one or more of such factors or factor types may be introduced. For example, multiple factors affecting therapeutic angiogenesis, including S1P, LPA, PA, VEGF, and/or any other drug or agent may be introduced at a single time.

Platelets are typically two to four microns (μm) in diameter. Thus, platelets (either modified or not) may be introduced through any micro-catheter or needle-type device that can access a target site thus permitting platelet delivery without causing cell lysis. In one embodiment, it is desired that platelets be delivered into the peri-adventitial space surrounding an artery (or vein) or an area radially outward from the peri-adventitial space by a needle-type device such as a transmural needle catheter system. Alternatively, the desired platelets may be delivered by a perfusion guide wire passed through, for example, an arterial wall. In still another embodiment, the platelets may be introduced into the myocardium through a catheter placed in a chamber of the heart. Representative catheters include a STILETO™ intraventricular needle catheter commercially available from Boston Scientific of Maple Grove, Minn. In still a further embodiment, the platelets may be applied or introduced at a target site during a surgical procedure, either as a slurry of platelets, or as a component of a natural or synthetic matrix. In yet a further embodiment, particularly applicable to the peripheral circulation, the platelets may be injected through a needle and/or catheter inserted percutaneously using direct vision or imaged guided methods such as fluoroscopy, coherence tomography (CT), ultrasound, magnetic resonance (MR), etc.

A suitable apparatus (a catheter assembly) is described in the following paragraphs for accurately locating a treatment agent comprising a platelet or platelets at a location in a blood vessel (preferably beyond the media layer) or in the peri-adventitial space adjacent to a blood vessel or radially outward from the peri-adventitial space, or at another tissue location such as the tissue of the myocardium. It is appreciated that a catheter assembly is one technique for introducing treatment agents and the following description is not intended to limit the application or placement of the treatment agent compositions described above.

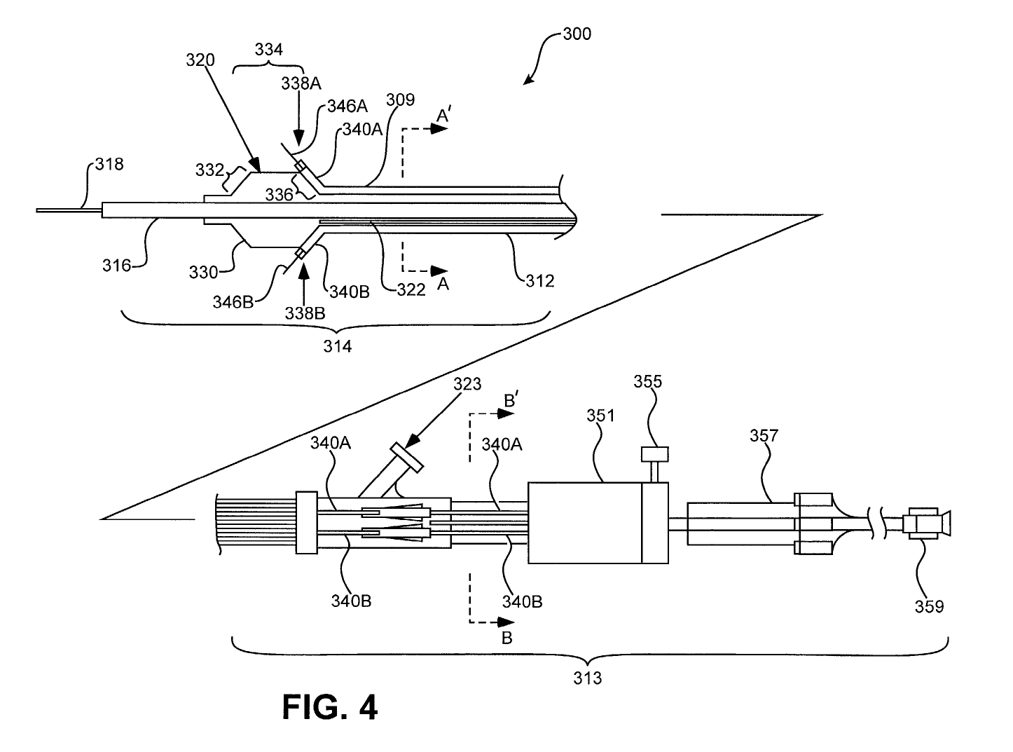

Referring now to the drawings, wherein similar parts are identified by like reference numerals, FIGS. 4, 5, and 6 illustrate one embodiment of a delivery apparatus. In general, the delivery apparatus provides a system for delivering a substance, such as a treatment agent of a composition comprising a platelet or a plurality of platelets or a combination of treatment agents, to or through a desired area of a blood vessel (a physiological lumen) or tissue in order to treat a localized area of the blood vessel or to treat a localized area of tissue possibly located adjacent to the blood vessel. The delivery apparatus is similar in certain respects to the delivery apparatus described in commonly-owned, U.S. patent application Ser. No. 09/746,498 (filed Dec. 21, 2000), titled “Directional Needle Injection Drug Delivery Device”, of Chow, et al., and incorporated herein by reference. The delivery apparatus includes a catheter assembly 300, which is intended to broadly include any medical device designed for insertion into a blood vessel or physiological lumen to permit injection and/or withdrawal of fluids, to maintain the potency of the lumen, or for any other purpose.

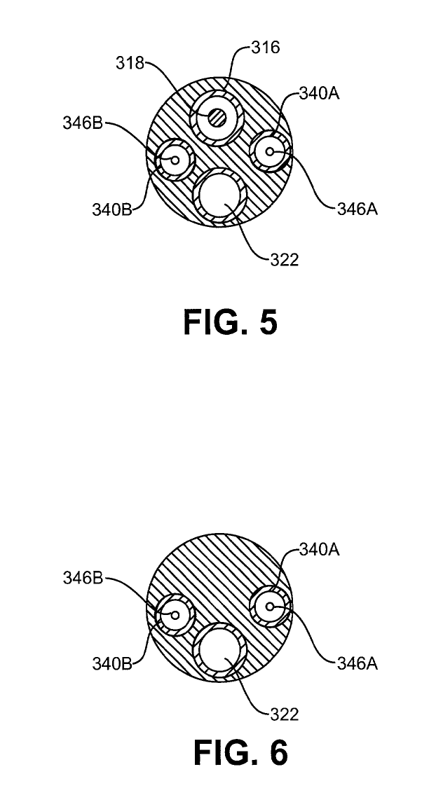

In one embodiment, catheter assembly 300 is defined by elongated catheter body (cannula) 312 having proximal end 313 and distal end 314. FIG. 5 shows catheter assembly 300 through line A-A′ of FIG. 4 (at distal end 314). FIG. 6 shows catheter assembly 300 through line B-B′ of FIG. 4 (at proximal end 313).

Referring to FIG. 4 and FIG. 5, catheter assembly 300 includes catheter body 312 extending from proximal end 313 to distal end 314. In this example, guidewire lumen 316 is formed within catheter body 312 for allowing catheter assembly 300 to be fed and maneuvered over guidewire 318 (shown at this point within guidewire lumen 316).

Balloon 320 is incorporated at distal end 314 of catheter assembly 300 and is in fluid communication with inflation lumen 322 formed within catheter body 312 of catheter assembly 300. Balloon 320 includes balloon wall or membrane 330 which is selectively inflatable to dilate from a collapsed configuration to a desired and controlled expanded configuration. Balloon 320 can be selectively dilated (inflated) by supplying a fluid into inflation lumen 322 at a predetermined rate of pressure through inflation port 323. Balloon wall 330 is selectively deflatable, after inflation, to return to the collapsed configuration or a deflated profile. In one embodiment, balloon wall 330 can be defined by three sections, distal taper wall 332, medial working length 334, and proximal taper wall 336. In one embodiment, proximal taper wall 336 can taper at any suitable angle 0, typically between about 100 to less than about 90°, when balloon 320 is in the expanded configuration.

Distal taper wall 332, medial working length 334, and proximal taper wall 336 of balloon wall 330 can be bound together by seams or be made out of a single seamless material. Balloon 320 can be made from any suitable material, including, but not limited to, polymers and copolymers of polyolefins, polyamides, polyesters and the like. The specific material employed must be mutually compatible with the fluids employed in conjunction with balloon 320 and must be able to stand the pressures that are developed within balloon 320. Balloon wall 330 can have any suitable thickness so long as the thickness does not compromise properties that are critical for achieving optimum performance. Such properties include high burst strength, low compliance, good flexibility, high resistance to fatigue, the ability to fold, the ability to cross and re-cross a desired region of treatment or an occluded region in a lumen, and low susceptibility to defects caused by handling. By way of example, and not limitation, the thickness can be in the range of about 10 microns to about 30 microns, the diameter of balloon 320 in the expanded configuration can be in the range of about 2 millimeters (mm) to about 10 mm, and the length can be in the range of about 3 mm to about 40 mm, the specific specifications depending on the procedure for which balloon 320 is to be used and the anatomy and size of the target lumen in which balloon 320 is to be inserted.

Balloon 320 may be dilated (inflated) by the introduction of a liquid into inflation lumen 322. Liquids containing therapeutic and/or diagnostic agents may also be used to inflate balloon 320. In one embodiment, balloon 320 may be made of a material that is permeable to such therapeutic and/or diagnostic liquids. To inflate balloon 320, the fluid can be supplied into inflation lumen 322 at a predetermined pressure, for example, between about one and 20 atmospheres.

Catheter assembly 300 also includes substance delivery assembly 338A and substance for injecting a treatment agent into a tissue of a physiological passageway. In one embodiment, delivery assembly 338A includes needle 346A having a lumen with a diameter of, for example, 0.004 inches (0.010 cm) to 0.012 inches (0.030 cm). Needle 346A is movably disposed within delivery lumen 340A formed in catheter body 312. Delivery assembly 338B includes needle 346B movably disposed within delivery lumen 340B formed in catheter body 312. Delivery lumen 340A and delivery lumen 340B each extend between distal end 314 and proximal end 313. Delivery lumen 340A and delivery lumen 340B can be made from any suitable material, such as polymers and copolymers of polyamides, polyolefins, polyurethanes, and the like. Access to the proximal end of delivery lumen 340A or delivery lumen 340B for insertion of needle 346A or 346B, respectively, is provided through hub 351.

One or both of delivery lumen 340A and delivery lumen 340B may be used to deliver a treatment agent to a treatment site (e.g., through needle 346A and/or needle 346B). Alternatively, one delivery lumen (e.g., delivery lumen 340A via needle 346A) may be used to deliver a treatment agent (e.g., therapeutic angiogenic treatment agent) while the other delivery lumen (e.g., delivery lumen 340B via needle 346B) may be used to deliver a therapeutic substance that is a non-therapeutic angiogenic substance.

Catheter assembly 300 may also include an imaging assembly. Suitable imaging assemblies include ultrasonic imaging assemblies, optical imaging assemblies, such as an optical coherence tomography (OCT) assembly, magnetic resonance imaging (MRI).

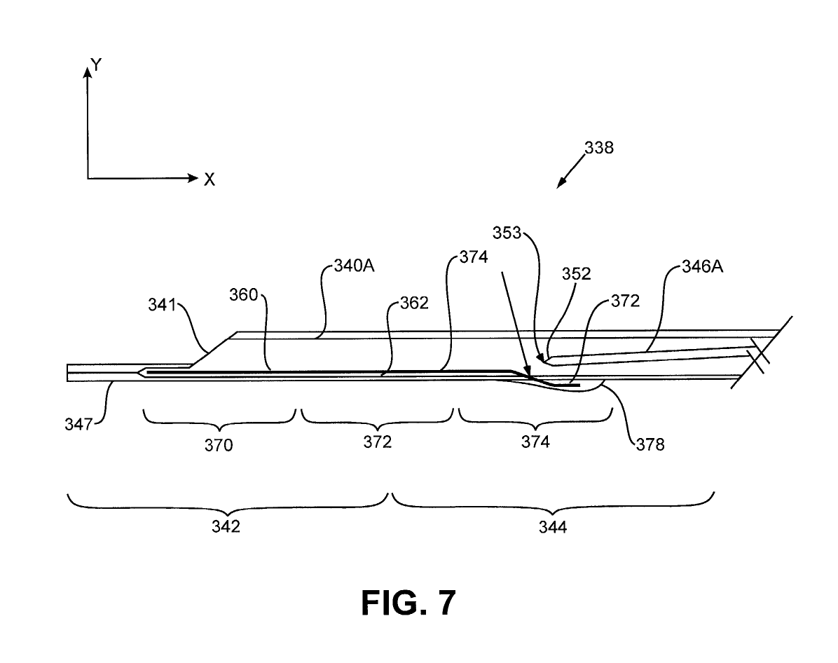

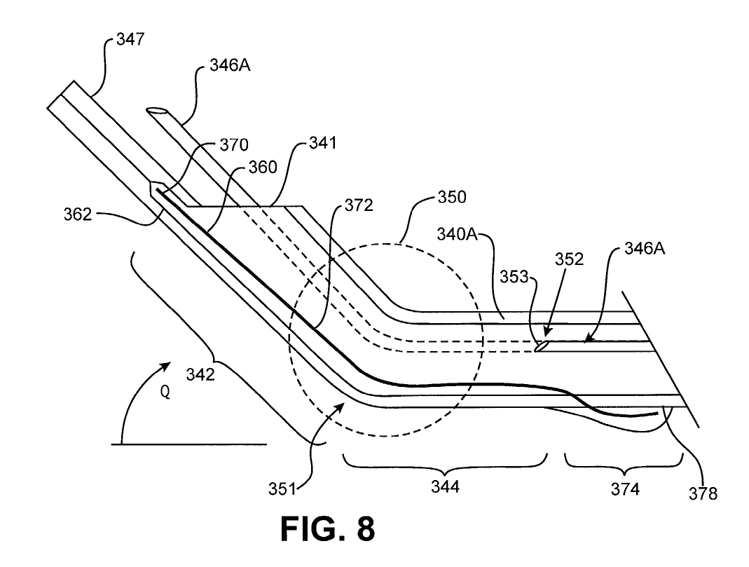

FIGS. 7 and 8 are simplified sectional views of therapeutic substance delivery assembly 338A in an undeployed and deployed arrangement, respectively. Delivery lumen 340A includes distal or first section 342 and proximal or second section 344. Distal section 342 can include overhang section 347 that extends beyond opening 341 to provide a means for securing delivery lumen 340A to balloon 320. For example, overhang section 347 can be adhered along the proximal taper wall 336 and working length 334 of balloon 320. In this manner, delivery lumen 340A is continually supported during, until, and after needle 346A is extended from delivery lumen 340A. In one embodiment, as shown in FIG. 8, delivery lumen 340A includes bend region 350 at which distal section 342 of delivery lumen 340A is capable of bending (or generally rotating) about pivotal point 351 with respect to proximal section 344. For example, to accomplish the pivotal movement, distal section 342 of delivery lumen 340A is in contact with proximal taper wall 336 of balloon 320 (FIG. 4). Accordingly, in response to the inflation of balloon 320, section 342 moves relative to section 344 to form bend region 350. In one embodiment, section 342 can move from a substantially longitudinal position to a substantially perpendicular position. Thus, the angle 0 of bend region 350 can vary between 0° and 90°. In one example, after inflation of balloon 320, angle 0 can range from between about 10° and 90°, for example, 45°.

Needle 346A is slidably or movably disposed in delivery lumen 340A. Needle 346A includes tissue-piercing tip 352 having dispensing port 353. Dispensing port 353 is in fluid communication with a lumen (not shown) of needle 346A. In one embodiment, the lumen of needle 346A can be pre-filled with a measured amount of a treatment agent. The lumen of needle 346A connects dispensing port 353 with treatment agent injection port 359 (FIG. 4), which is configured to be coupled to various substance dispensing means of the sort well known in the art, for example, a syringe or fluid pump. Injection port 359 allows a measured treatment agent to be dispensed from dispensing port 353 as desired or on command.

Needle 346A is coupled at proximal end 313 of catheter assembly 310 in a needle lock 355 (FIG. 4). Needle lock 355 can be used to secure needle 346A in position once needle 346A has been either refracted and/or extended from delivery lumen 340A as described below. In one embodiment, an adjustment knob 357 can be used to set the puncture distance of needle 346A as it is extended out from delivery lumen 340A and into the wall of the physiological lumen. For example, adjustment knob 357 may have calibrations, such that each revolution of the adjustment knob from one calibrated mark to another represents a fixed distance of travel for needle 346A. The portion of needle 346A protruding from delivery lumen 340 can be of any predetermined length, the specific length being dependent upon the desired depth of calibrated penetration and the procedure for which delivery assembly 338A is to be used. The protruding length of needle 346A can be from about 250 microns to about four cm. It is appreciated that other mechanisms for securing needle 346A at a retracted or extended position may alternatively be used, including the incorporation of a mechanical stop optionally including a signaling (e.g., electrical signaling) device as described in commonly-owned U.S. patent application Ser. No. 09/746,498 (filed Dec. 21, 2000), titled “Directional Needle Injection Drug Delivery Device”, and incorporated herein by reference.

Needle 346A is slidably disposed in delivery lumen 340A, so that it can move between a first retracted position (FIG. 7) and a second extended position (FIG. 8). In its first refracted position, tissue-piercing tip 352 is located inboard of the distal surface of catheter body 312, so as to avoid damaging tissue during deployment of catheter assembly 310. In its second or extended position, tissue-piercing tip 352 is located outboard of the distal surface of catheter body 312, so as to permit needle tip 352 to penetrate the tissue surrounding the physiological passageway in which catheter assembly 310 is disposed.

Referring again to FIGS. 7 and 8, deflector 360 is disposed along an inner wall 362 of delivery lumen 340A. In one embodiment, deflector 360 includes distal section 370, medial section 372 and proximal section 374. In one embodiment, distal section 370 can be supported by delivery lumen 340A by bonding distal section 370 to overhang section 347 of delivery lumen 340A. Medial section 372 of deflector 360 can be disposed on inner wall 362 of delivery lumen 340A, such that as delivery lumen section 342 rotates relative to delivery section 344 to form bend region 350, deflector 360 is positioned over the outside of the curvature of bend region 350. Proximal section 374 exits out of delivery lumen 340A and is adhered to an outside wall 378 of delivery lumen 340A using an adhesive, such as glue or the like.

Deflector 360 can be any device that will provide a shield to protect the wall of delivery lumen 340A while being small enough, such that deflector 360 does not impact the track of catheter assembly 310 in any significant manner. In one embodiment, deflector 360 can be a ribbon member. The ribbon member can be made thin, flexible and resilient such that the ribbon member can move and bend as delivery lumen sections 342 and 344 bend and move relative to each other. Positioning deflector 360 of a ribbon member on the outside of the curvature of bend region 350 allows deflector 360 to shield the delivery lumen wall from piercing and the like by needle 346A as needle 346A moves through bend region 350. Deflector 360 also provides a surface upon which needle 346A can be made to track through bend region 350.

Deflector 360 is sized to fit into and along inner wall 362 of delivery lumen 340A without occluding or interfering with the ability of needle 346A to translate through bend region 350. For example, deflector 360 can have a thickness of between about 0.0005 inches (0.127 mm) and about 0.003 inches (0.762 mm). The width of deflector 360 may be between about 0.005 inches (1.27 mm) and about 0.015 inches (3.81 mm). The length of deflector 360 may be between about 1 cm and about 10 cm. Deflector 360 can be made from any suitable material which allows deflector 360 to function, such as stainless steel, platinum, aluminum and similar alloy materials with similar material properties. In one embodiment, deflector 360 can be made from super-elastic alloys, such as nickel titanium alloys, for example NiTi.

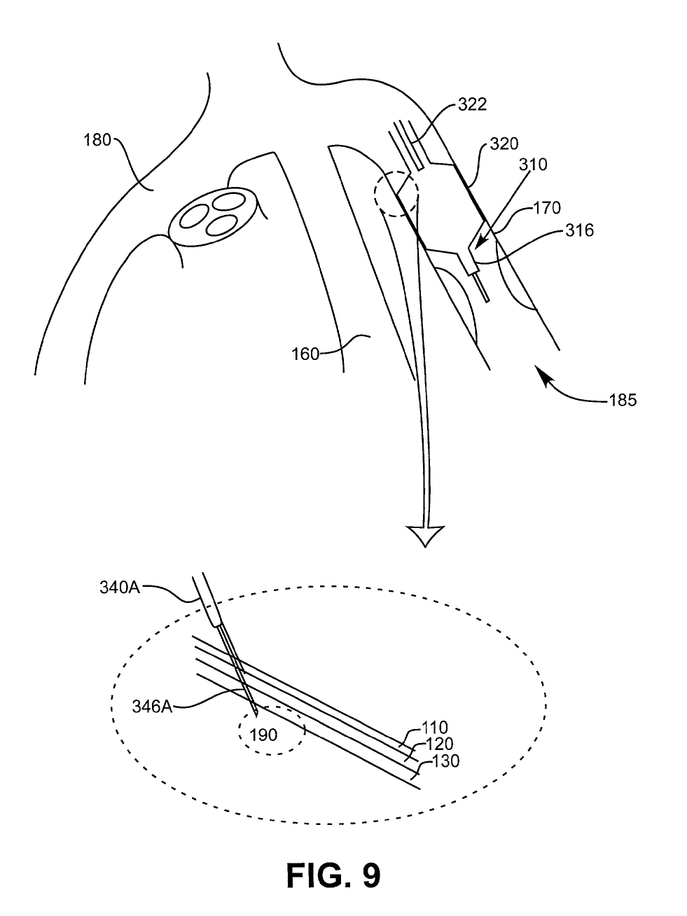

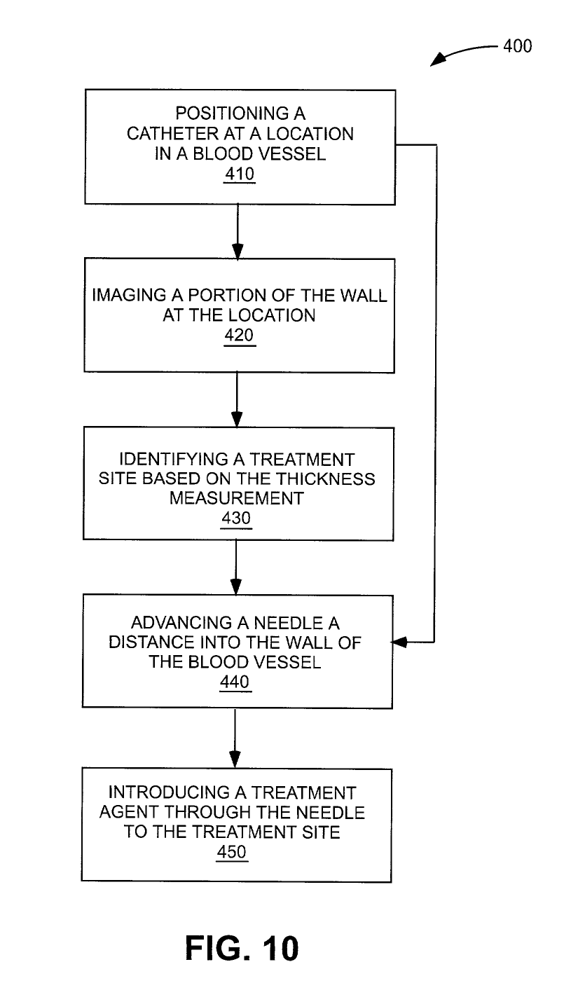

The catheter assembly described with reference to FIG. 3 may be used to introduce a treatment agent (e.g., a composition comprising a platelet or a plurality of platelets) such as described above at a desired location. FIG. 9 illustrates one technique. FIG. 10 presents a block diagram of one technique. With reference to FIGS. 9 and 10, guidewire 318 is introduced into, for example, arterial system of the patient (e.g., through the femoral artery) until the distal end of guidewire 318 is upstream of the narrowed lumen of the blood vessel (e.g., upstream of occlusion 185). Catheter assembly 300 is mounted on the proximal end of guidewire 318 and advanced over the guidewire 318 until catheter assembly 300 is positioned as desired. In the example shown in FIG. 9, catheter assembly 310 is positioned so that balloon 320 and delivery lumen 340a are upstream of the narrowed lumen of LCX 170 (block 410). Angiographic or fluoroscopic techniques may be used to place catheter assembly 300. Once balloon 320 is placed, a treatment site of the blood vessel may be identified by further imaging techniques, including but not limited to, optical coherence tomography, ultrasonic, or magnetic resonance techniques. An example of an optical imaging technique is described in co-pending commonly-assigned U.S. patent application Ser. No. 10/011,071, and subject to low inflation pressure, guidewire 318 is removed and replaced in one embodiment with an optical fiber. In the catheter assembly shown in FIG. 9, the imaging portion of an imaging device (e.g., OCT, ultrasonic, etc.) may be within the imaging lumen as the catheter is positioned. Once positioned, in this case upstream of occlusion 185, the imaging assembly is utilized to view the blood vessel and identify the various layers of the blood vessel (block 420).

The imaging assembly provides viewable information about the thickness or boundary of the intimal layer 110, media layer 120, and adventitial layer 130 of LCX 170 (See FIG. 1). The imaging assembly may also be used to measure a thickness of a portion of the blood vessel wall at the location, e.g., the thickness of the various layers of LCX 170.

LCX 170 is viewed and the layer boundary is identified or a thickness of a portion of the blood vessel wall is imaged (and possibly measured) (block 140). The treatment site may be identified based on the imaging (and possibly measuring). In one example, the treatment site is a peri-adventitial site (e.g., site 190) adjacent to LCX 170. At this point, balloon 320 is dilated as shown in FIG. 7 by, for example, delivering a liquid or gas to balloon 320 through inflation lumen 322. The inflation of balloon 320 causes needle lumen 338 to move proximate to or contact the blood vessel wall adjacent to the treatment site. Needle 346A is then advanced a distance into the wall of the blood vessel (block 140). A real time image may be used to advance needle 346A. Alternatively, the advancement may be based on a measurement of the blood vessel wall or layer boundary derived from an optical image.

In the embodiment shown in FIG. 10, needle 346A is advanced through the wall of LCX 170 to peri-adventitial site 190. Needle 346A is placed at a safe distance, determined by the measurement of a thickness of the blood vessel wall and the proximity of the exit of delivery lumen 340A to the blood vessel wall. Adjustment knob 357 may be used to accurately locate needle tip 346A in the desired peri-adventitial region. Once in position, a treatment agent is introduced through needle 346A to the treatment site (e.g., peri-adventitial site 190).

In the above described embodiment of locating a treatment agent within or beyond a blood vessel wall (e.g., at a peri-adventitial site), it is appreciated that an opening is made in or through the blood vessel. In some instances, it may be desirable to plug or fill the opening following delivery of the treatment agent. This may be accomplished by introduction through a catheter lumen of cyanoacrylate or similar material that will harden on contact with blood.

In the above-described embodiment of locating a treatment agent within or beyond a blood vessel wall (e.g., at a peri-adventitial site), imaging techniques were described to locate the treatment site. It is appreciated that the treatment site may be selected under direct vision through, for example, surgical techniques. With reference to FIG. 10, for example, such a technique would involve, according to one example, positioning a catheter comprising a treatment agent including modified or unmodified platelets as described above in a blood vessel (block 410), advancing a needle of the catheter into or beyond the wall of the blood vessel (block 440), and delivering the treatment agent (block 450).

In the above embodiment, an illustration and method was described to introduce a treatment agent at a peri-adventitial site. It is appreciated that the treatment agent may be introduced to a portion of the wall of the blood vessel. In another embodiment, the introduction is at a point beyond the media layer (e.g., beyond media layer 120 in FIG. 1) to the adventitial layer (e.g., adventitial layer 130 in FIG. 1). Further, the techniques and treatment agents described may further be used to introduce a treatment agent directly into the tissue of the myocardium.

In the preceding detailed description, the invention is described with reference to specific embodiments thereof. It will, however, be evident that various modifications and changes may be made thereto without departing from the broader spirit and scope of the invention as set forth in the claims. The specification and drawings are, accordingly, to be regarded in an illustrative rather than a restrictive sense.

In connection with the description of the various exemplary embodiments, the following definitions are utilized:

“Therapeutic angiogenesis” refers to the processes of causing, inducing, or modulating angiogenesis and arteriogenesis.

“Angiogenesis” is the promotion or causation of the formation of new blood vessels.

“Arteriogenesis” is the formation of collateral vessels, which typically occurs in a non-ischemic region of a vessel. The collateral vessels allow blood to flow from a well-perfused region of the vessel into the ischemic region.

“Ischemia” is the localized reduction in blood flow caused by narrowing or occlusion of one or more vessels, such as coronary arteries or their branches, most often through thrombosis or via deposits of fat, connective tissue, calcification of the walls, or restenosis caused by the abnormal migration and proliferation of smooth muscle cells.

“Occlusion” is defined as the total or partial obstruction of blood flow through a vessel.

“Treatment agent” includes, but is not limited to, agents directed to specific binding sites (e.g., receptor binding treatment agents).

“Specific binding treatment agent” or “receptor binding treatment agent” includes a protein or small molecule that will induce and/or modulate a therapeutic angiogenic response. Representative treatment agents include, but are not limited to, vascular endothelial growth factor (VEGF) in any of its multiple isoforms, fibroblast growth factors, monocyte chemoattractant protein 1 (MCP-1), transforming growth factor beta (TGF-beta) in any of its multiple isoforms, transforming growth factor alpha (TGF-alpha), lipid factors, hypoxia-inducible factor 1-alpha (HIF-1-alpha), PR39 peptides, DEL 1, nicotine, insulin-like growth factors, placental growth factor (P1GF), hepatocyte growth factor (HGF), estrogen, follistatin, proliferin, prostaglandin E1, prostaglandin E2, cytokines, tumor necrosis factor (TNF-alpha), erythropoietin, granulocyte colony-stimulating factor (G-CSF), granulocyte macrophage colony-stimulating factor (GM-CSF), angiogenin, hormones, and genes that encode such substances.

The above description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

What is claimed is:

1. A method for inhibiting heart failure after myocardial infarction, the method comprising:
obtaining platelets from a donor;
increasing stromal cell derived factor 1 (SDF-1) content of the donor platelets; and
administering the platelets to the heart of a subject.

2. The method of claim 1, wherein the donor is a mammal.

3. The method according to claim 2, wherein the mammal is a human.

4. The method of claim 1, wherein the SDF-1 content is increased by electroporating the donor platelets in the presence of SDF-1.

5. The method of claim 1, wherein the platelets are administered to the myocardium.

6. The method of claim 5, wherein the myocardium is infarcted.

7. The method of claim 1, wherein the platelets are activated with a cytokine prior to administration.

8. The method of claim 7, wherein the cytokine is thrombopoietin.

9. The method according to claim 8, wherein the cytokine is a soluble kit ligand.

10. The method of claim 1, wherein the platelets are administered to a coronary artery.

11. The method of claim 10, wherein the coronary artery supplies an infarcted myocardium.

12. The method of claim 1, wherein the platelets are administered into the left ventricular chamber, or by intra-arterial or intravenous injection.

13. The method of claim 12, wherein the platelets bind to the surfaces of injured arteries within the infarct zone after delivery.

14. The method of claim 12, wherein the methods promote aggregation of platelets to form microvascular obstructions in the infarct zone after delivery.

15. The method according to claim 12, wherein the method promotes stimulation of progenitor cell recruitment and sequestration to replace myocardial cells and promote revascularization of the infarct site.

16. A composition comprising:
(a) modified donor platelets, wherein the modified donor platelets have a greater concentration of stromal cell derived factor 1 than unmodified donor platelets, and
(b) a growth factor.

17. The composition of claim 16, wherein the composition further includes a cytokine

18. The composition of claim 17, wherein the cytokine is thrombopoietin.

19. The composition of claim 16, wherein the composition promotes stimulation of progenitor cell recruitment and sequestration.

20. The composition of claim 16, wherein the composition promotes revascularlization.
